(19) United States
(12) Patent Application Publication
(10) Pub. No.: US 2017/0049356 A1
(43) Pub. Date: Feb. 23, 2017

(54) An Apparatus and Method for "High-Resolution" Electrical Impedance Imaging

(71) Applicant: Wei Wang, Leicester (GB)

(72) Inventor: Wei Wang, Leicester (GB)

(21) Appl. No.: 15/119,157

(22) PCT Filed: Feb. 16, 2015
(86) PCT No.: PCT/GB2015/050432

§ 371 Date: Aug. 16, 2016

(30) Foreign Application Priority Data
Feb. 16, 2014 (GB) 1402701.5

(51) Int. Cl. A61B 5/053

(52) U.S. Cl. CPC A61B 5/0536; A61B 2562/0209

(57) ABSTRACT

A method of high resolution electrical impedance imaging including using an array of sampling points defined by an electrode frame at a first position, wherein the electrode frame defines a relative displacement of sampling points; and using a different array of sampling points defined by the same electrode frame at a different, second position.

### FIELD OF THE INVENTION

Embodiments of the present invention relate to an apparatus and method for “high-resolution” electrical impedance imaging.

### BACKGROUND TO THE INVENTION

Electrical impedance mammography (EIM), or Electrical impedance imaging (EII), also referred to as electrical impedance tomography (EIT), electrical impedance scanner (EIS) and applied potential tomography (APT), is an imaging technique that is particularly used in medical applications.

The technique images the spatial distribution of electrical impedance inside an object, such as the human body. The technique is attractive as a medical diagnostic tool because it is non-invasive and does not use ionizing radiation as in X-ray tomography or the generation of strong, highly uniform magnetic fields as in Magnetic Resonance Imaging (MRI).

Typically a two-dimensional (2D) or three-dimensional (3D) array of evenly spaced electrodes is attached to the object to be imaged about the region of interest. Either input voltages are applied across pairs of ‘input’ electrodes and output electric currents are measured at the ‘output’ electrodes or input electric currents are applied between pairs of ‘input’ electrodes and output voltages are measured between at the ‘output’ electrodes or between pairs of output electrodes. For example, when a very small alternating electric current is applied between a pair of ‘input’ electrodes, the potential difference between all other pairs of ‘output’ electrodes is measured. The current is then applied between a different pair of ‘input’ electrodes and the potential difference between all other pairs of ‘output’ electrodes is measured. An image is constructed using an appropriate image reconstruction technique.

Spatial variations revealed in electrical impedance images may result from variations in impedance between healthy and non-healthy tissues, variations in impedance between different tissues and organs or variations in apparent impedance due to anisotropic effects resulting for example from muscle alignment.

Tissue or cellular changes associated with cancer cause significant localized variations in electrical impedance and can be imaged. WO 00/12005 discloses an example of electrical impedance imaging apparatus that can be used to detect breast carcinomas or other carcinomas.

### BRIEF SUMMARY

According to various, but not necessarily all, embodiments of the invention there is provided methods, apparatus and computer programs as defined in the appended claims.

These embodiments enable “higher” resolution images than traditional electrical impedance imaging.

### BRIEF DESCRIPTION

For a better understanding of various examples that are useful for understanding the brief description, reference will now be made by way of example only to the accompanying drawings in which:

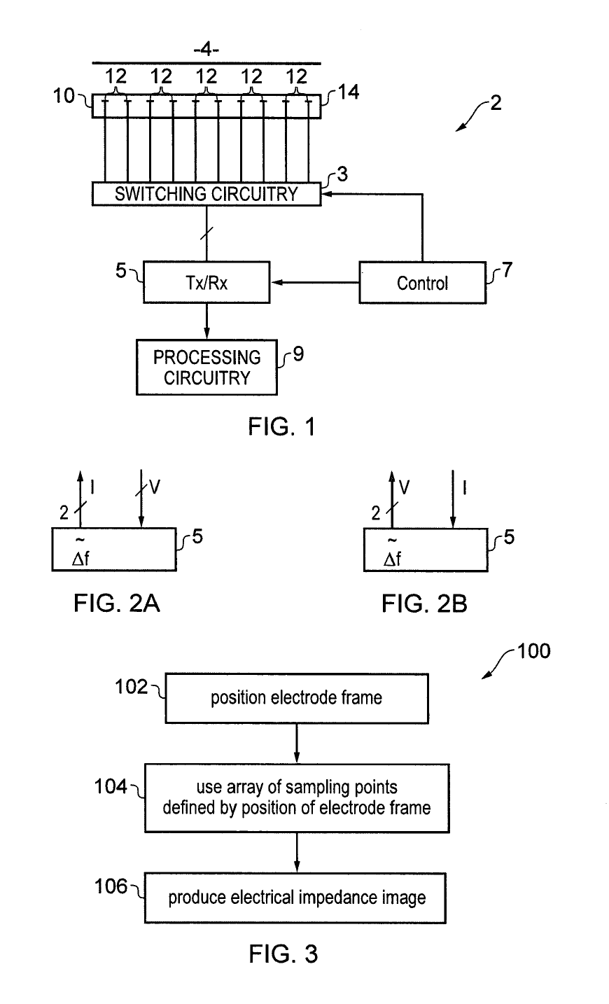

FIG. 1 illustrates an example of an apparatus which is suitable for performing electrical impedance imaging;

FIGS. 2A and 2B illustrate examples of the transceiver circuitry;

FIG. 3 illustrates a method;

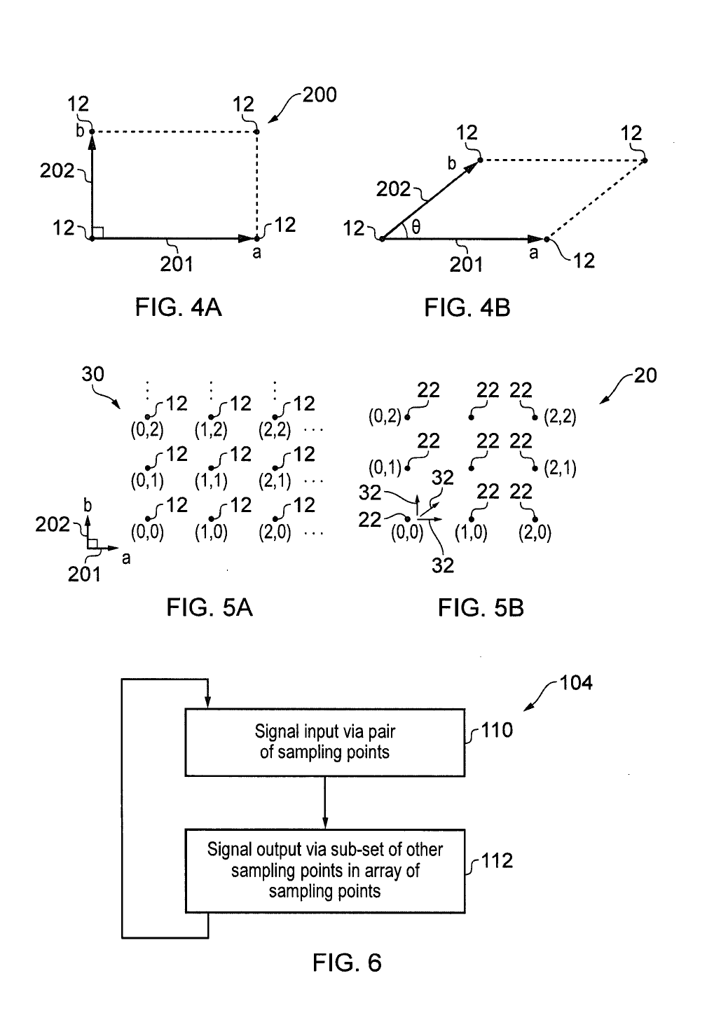

FIGS. 4A and 4B illustrate different unit cells that are tessellated to form an electrode frame;

FIGS. 5A and 5B illustrate that the electrode frame may be repositioned using defined positional offsets;

FIG. 6 illustrates an example of a method;

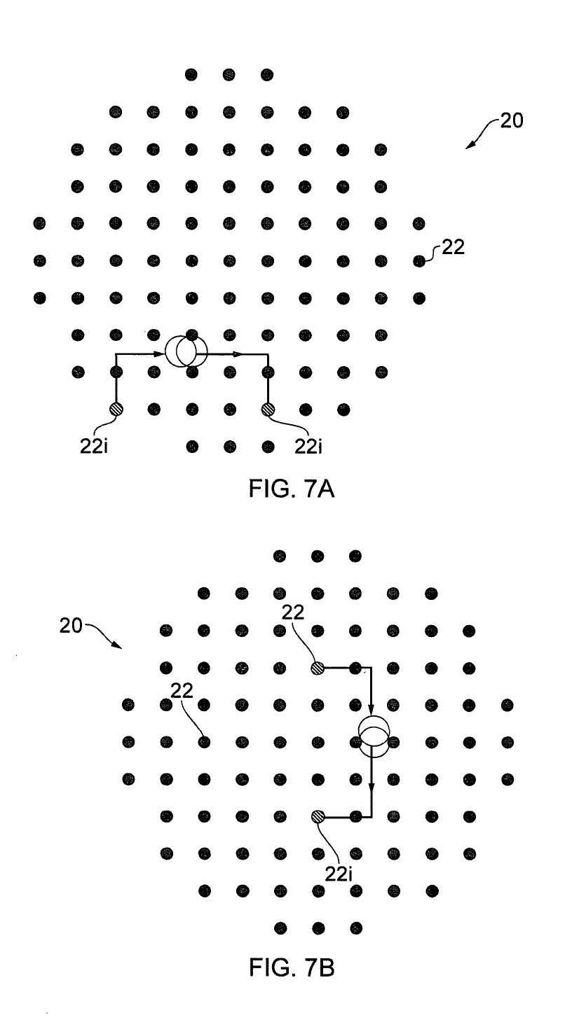

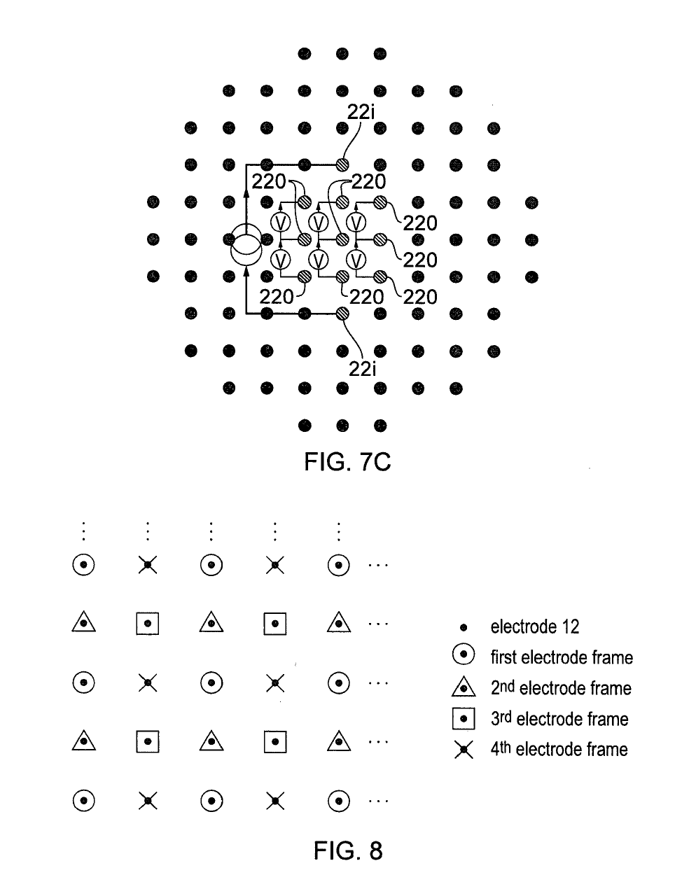

FIG. 7A or 7B illustrate an example where an input signal is an electric current applied between the pair of sampling points;

FIG. 7C illustrates an example where voltage differences between adjacent pairs of sampling points are measured;

FIG. 8 illustrates an example of how an electrode frame may be (re)positioned without (re)positioning the electrode array;

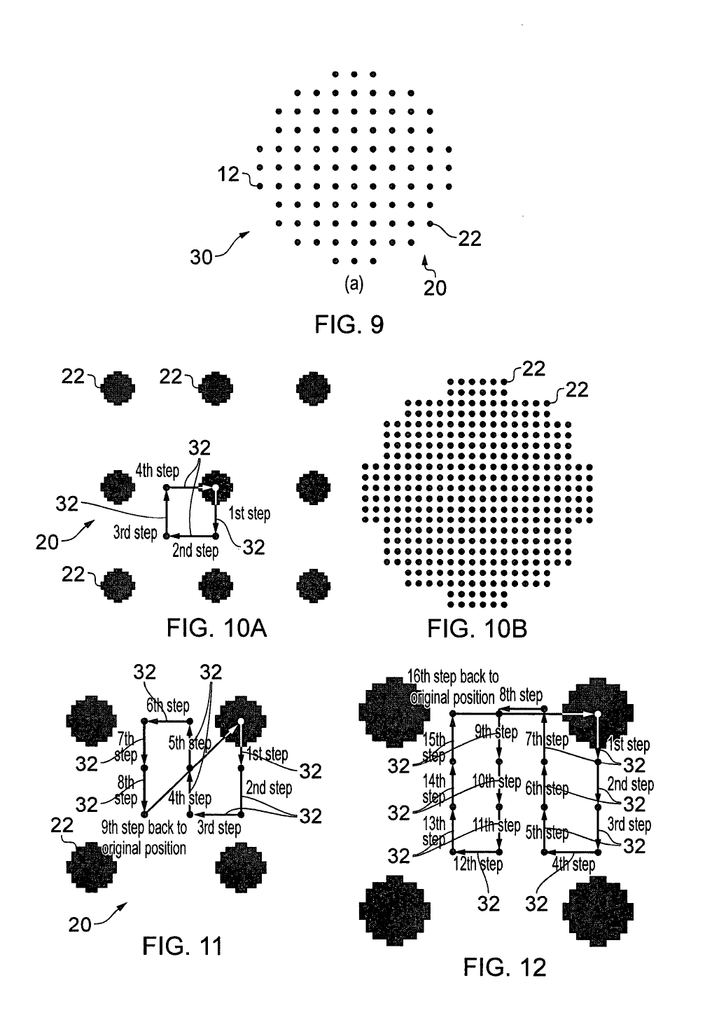

FIGS. 9 to 12 illustrate examples of how an electrode frame may be (re)positioned by (re)positioning the electrode array

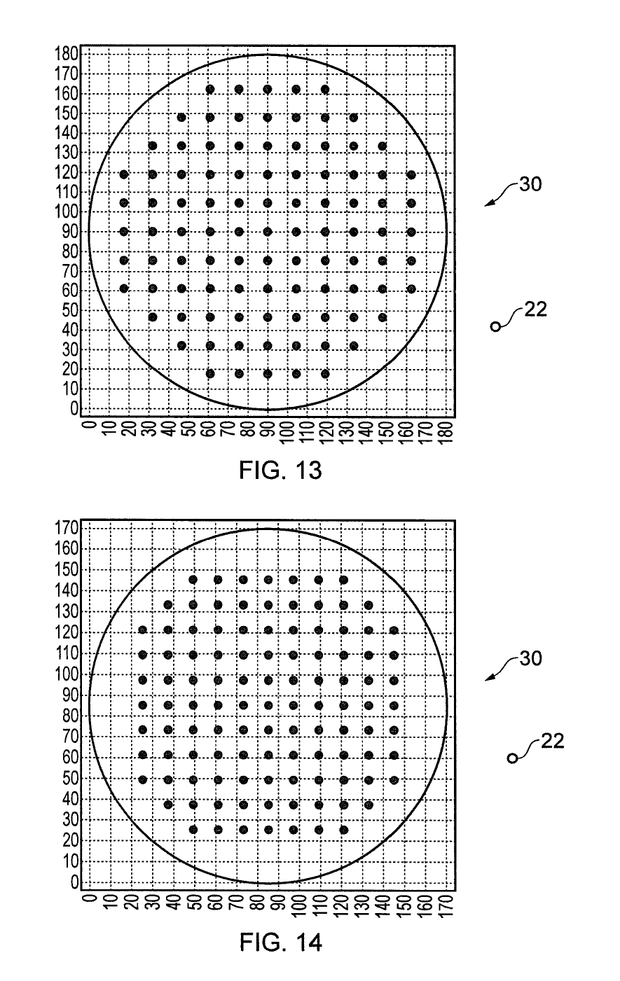

FIGS. 13 and 14 illustrate different examples of “square electrode” based frames;

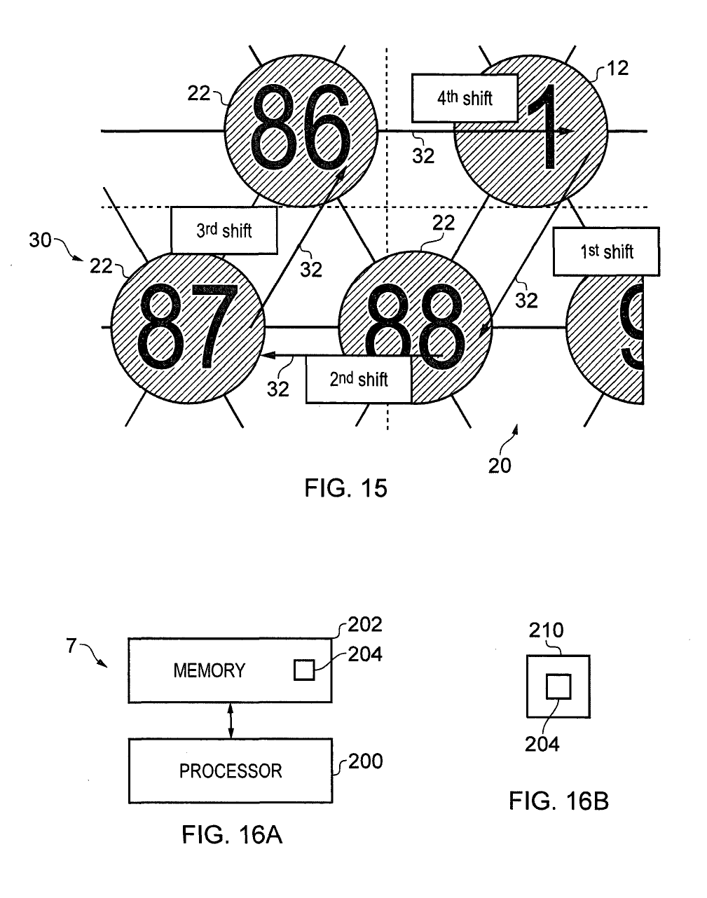

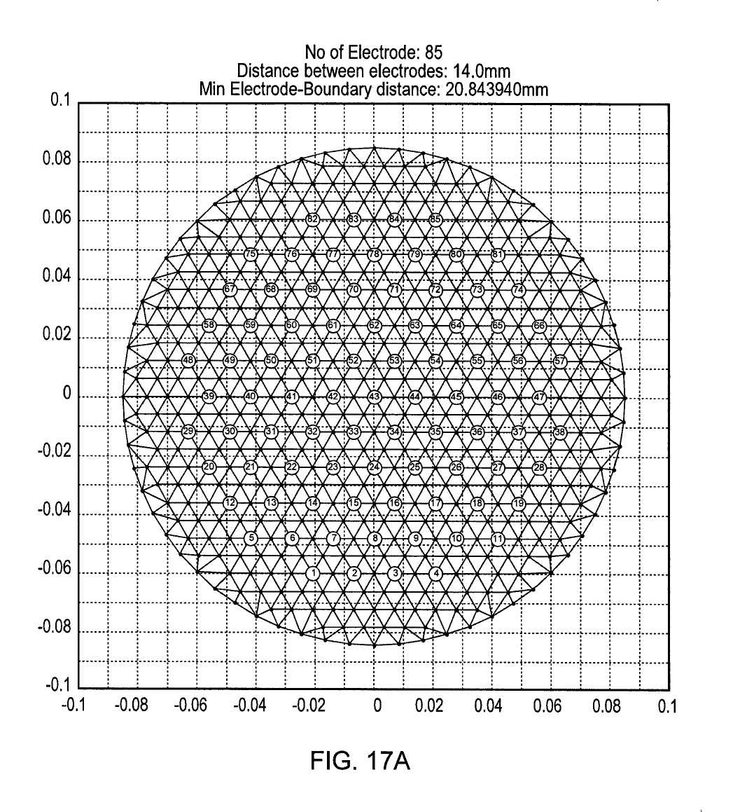

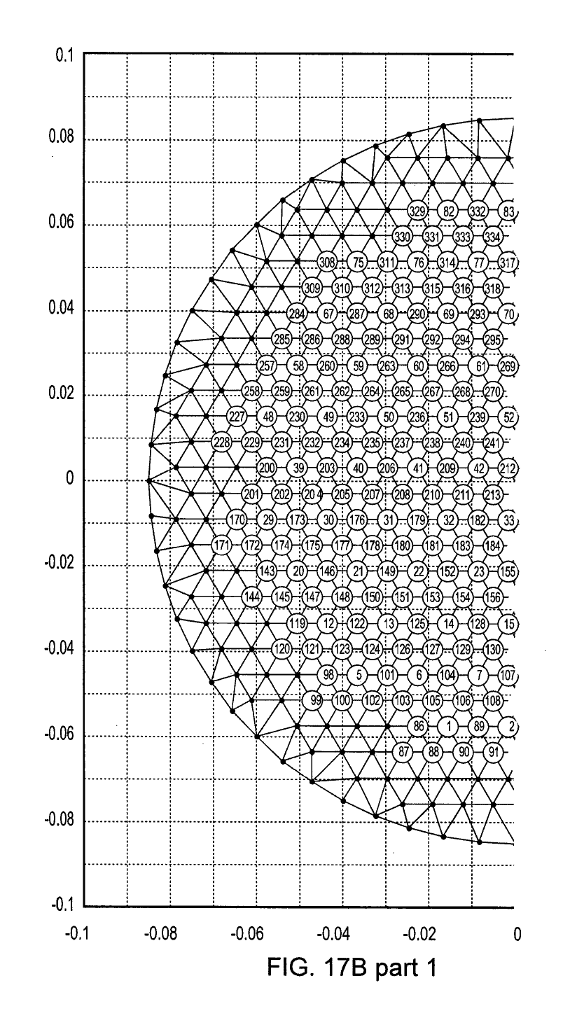

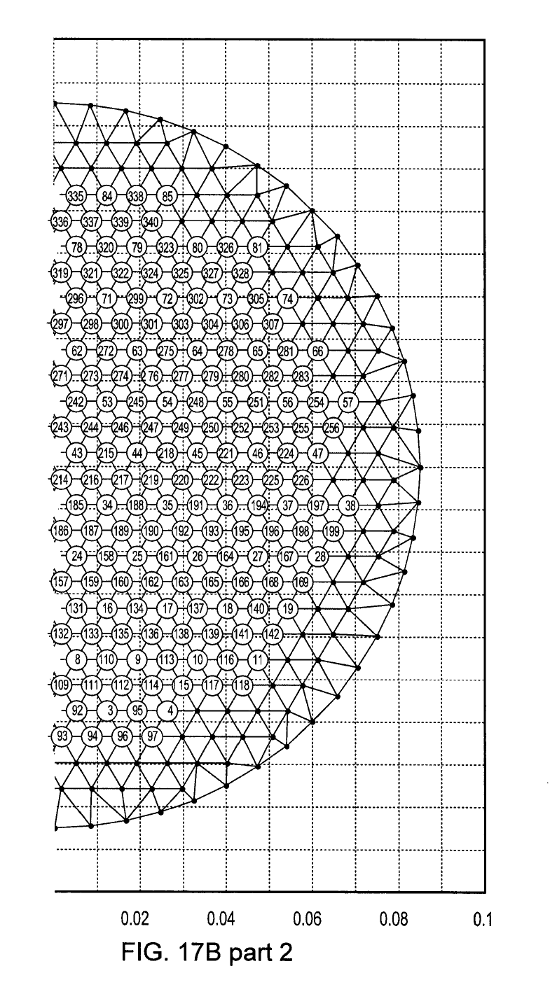

FIGS. 15, 17A and 17B illustrate a further example of how an electrode frame may be (re)positioned by (re)positioning a “triangle electrode array”

FIG. 16A illustrates an example of controlling circuitry; and

FIG. 16B illustrates a delivery mechanism for a computer program.

### DETAILED DESCRIPTION

In the following description reference will be made to electrodes 12, an electrode array 10 comprising a plurality of electrodes, an electrode frame 30, an array 20 of sampling points 22 and a sub-set of sampling points 22. It will be instructive to clarify, at an early stage, the similarities and differences between these terms.

An electrode 20 is a physical, conductive electrode that is used to either provide an electrical signal and/or to receive an electrical signal. The electrode array 10 is a physical arrangement of the electrodes 12 in space. This arrangement is most commonly fixed such that the electrodes 12 have a fixed spatial relationship relative to each other.

A sampling point 22 is a point that corresponds to an electrode 12 and which may be used to provide an electrical signal and/or receive an electrical signal. The array 20 of sampling points 22 defines the sampling points 22 that are available for sampling at that time. The array of sampling points is determined by a position of an electrode frame 30 in space.

An electrode frame 30 defines the relative arrangement of sampling points 22 in space. The electrode frame 30 may be fixed such that the sampling points 22 have a fixed spatial relationship relative to each other. The electrode frame 30 may, however, be repositioned.

A sub-set of sampling points 22 represents some, but not all, of the array 20 of sampling points 22. Different sub-sets of sampling points are typically used in different time slices to cover a whole of an array of sampling points.

In a first embodiment, which may conveniently be referred to as the “virtual repositioning embodiment”, the electrode frame 30 is a sub-set of the electrode array 10. The electrode frame 30 may be defined by selecting a sub-set of the electrodes 12 of the electrode array 10. The position of the electrode frame 30 may be changed by selecting a different sub-set of the electrodes 12 of the electrode array 10.

In contrast, in a second embodiment which may be conveniently referred to as “the physical repositioning embodiment”, the electrode frame 30 is the same as the electrode array 10. There is a one-to-one correspondence between the electrodes 12 of the electrode array 10 and the sampling points 22 of the electrode frame 30. The different arrays 20 of sampling points 22 are defined by different physical positions of the electrode array 10 (electrode frame 30) and physical changes to the position of the electrode array 10 change the position of the electrode frame 30 and therefore change the array of sampling points 22.

In both the first and second embodiments of the invention, electrical impedance imaging is achieved by using an array 20 of sampling points 22 defined by an electrode frame 30 at a first position, wherein the electrode frame 30 defines the relevant displacement of sampling points 22; and by using a different array 20 of sampling points 22 defined by the same electrode frame 30 at a different second position.

It will be appreciated that in the first embodiment, the change in the array 20 of sampling points 22 is achieved by changing which electrodes 12 are used in the electrode frame 30 and in the second embodiment, the change in the array 20 of sampling points 22 is achieved by changing a physical position of the electrode frame 30 (electrode array 10).

FIG. 1 illustrates an example of an apparatus 2 which is suitable for performing electrical impedance imaging.

The apparatus 2 comprises an electrode array 10 comprising a plurality of electrodes 12. The electrodes 12 are typically supported by a substrate 14. The electrodes 12 may be recessed relative to a surface of the substrate 14. The electrodes 12 are used to provide an electrical signal to a body of a subject 4 and to receive in response electrical signals.

In some examples, a conductive liquid such as saline solution and/or a support, comprising thin conductivity matched material may be used to mediate between the electrode 12 and the body 4. The support may be used to support the body and avoid moving artefacts.

The electrode array 10 is, in this example, a planar array and the electrodes 12 lie within a single flat plane.

Switching circuitry 3 is used to control which of the plurality of electrodes 12 are used to provide an input signal produced at the transceiver 5 to the body 4 and to control which of the plurality of electrodes 12 are used to provide an electrical signal, in reply, from the body 4 to the transceiver circuitry 5.

The switching circuitry 3 may be controlled by control circuitry 7. In addition, the control circuitry 7 may also control the transceiver circuitry 5.

The transceiver circuitry 5 provides the signals received from the electrodes 12 to processing circuitry 9 where the electrical signals are processed to produce an electrical impedance image.

As illustrated in FIGS. 2A and 2B, the transceiver circuitry 5 and switching circuitry 3 typically work in combination to provide an input electrical signal to a pair of electrodes 12 and to receive in reply electrical signals from a plurality of electrodes 12. The provided electrical signal may be an alternating signal and the frequency of the provided electrical signal may be controlled by the control circuitry 7. The frequency may, for example, vary between 100 Hz and 10 MHz. The input electric signal typically comprises a plurality of different frequencies and at least some frequencies above 1 MHz. Frequencies from 100 Hz to above 1 MHz (preferably up to 10 MHz) have been used with the frequency bandwidth exceeding 1 MHz.

The total impedance of a tissue or group of cells can be modelled as a parallel intra-cellular impedance and a parallel extra-cellular impedance. The intra-cellular impedance can be modelled as a series connection of a capacitance Ci and a resistance Ri. The extra-cellular impedance can be modelled as a resistance Rx. At lower frequencies the total impedance is dominated by Rx and at higher frequencies the total impedance is dominated Ri//Rx. The frequency response is sensitive to variations in Ci, Ri and Rx and can be used to identify the presence of abnormal tissue.

In the example of FIG. 2A, the transceiver circuitry 5 provides electrical signals in the form of electrical current and receives electrical signals from the electrodes in the form of detected voltages. In the example of FIG. 2B, the transceiver circuitry provides the input electrical signals as voltages and receives electrical signals from the same or different electrodes in the form of electric current.

FIG. 3 illustrates a method 100 that may be performed by the apparatus 2.

The method 100 is a method of electrical impedance imagery. At block 102, the method 100 positions an electrode frame 30. The electrode frame 30 defines a fixed relative displacement of sampling points 22. Positioning the electrode frame defines an array 20 of sampling points 22. For example, if the electrode frame 30 is positioned in a first position, the electrode frame 30 defines a first array of sampling points 22.

The array of sampling points 22, defined by the position of the electrode frame 30, is then used for electrical impedance measurement.

The method then returns to block 102 where the position of the electrode frame 30 is changed to a new second position. The new second position of the electrode frame 30 defines a new second array 20 of sampling points 22 which are used for electrical impedance measurement. The method then again proceeds to block 104 where the new, different array of the sampling points 22, defined by the new position of the electrode frame, is used for electrical impedance measurement. The method can repeat a number of times using a plurality of different arrays 20 of sampling points 22 defined by different positions of the same electrode frame 30, to produce different sets of electrical impedance measurement data

At block 106, the electrical impedance measurement data for each of the different arrays 20 of sampling points 22 are used to produce an electrical impedance image. It will be appreciated that the number of and density of sampling points 22 used to produce this image is greater than a number of and density of sampling points 22 that would be used if only a single array 20 of sampling points 22 is used. Thus the electrical impedance image produced has a higher resolution.

It will therefore be appreciated that the repositioning of the electrode frame 30 to define different arrays 20 of sampling points 22 may be used to produce electrical impedance images of higher resolution.

An electrode frame 30 may be defined by tessellated unit cells 200 of electrodes 12. FIGS. 4A and 4B illustrate different examples of possible unit cells 200 of electrodes 12.

Each unit cell 200 is defined by a first basis vector a 201 and a second basis vector b 202. Four positions of the electrodes 12 of the unit cell 200 are defined by (0, 0), (1, 0), (0, 1), (1, 1) in the co-ordinate space defined by the first basis vector 201 and the second basis vector 202.

The tessellation of the unit cells 200 produces the electrode frame 30. Each of the positions of the electrodes 12 defined by the tessellated unit cells 200 define a sampling point 22 in the array 20 of sampling points 22.

In the example of FIG. 4A, the first basis vector 201 and the second basis vector 202 are orthogonal and the unit cell 200 is rectangular or square. In the example of FIG. 4B, the first basis vector 201 and the second basis vector 202 are non-parallel and the unit cell 200 is a parallelogram. In some but not necessarily all examples, the angle θ between the first basis vector 201 and the second basis vector 202 may be 60°.

In some, but not necessarily all, examples the magnitude of the first basis vector 201 and the magnitude of the second basis vector 202 may be the same such that, for example, the unit cell 200 in FIG. 4A is a square and the unit cell in FIG. 4B is a rhombus.

FIGS. 5A and 5B illustrate that the electrode frame 30 may be repositioned using defined positional offsets 32.

In this example, the offsets are linear translations defined with respect to the first basis vector 201 and the second basis vector 202. However, in other examples the offsets may be rotations of the unit cell 200.

An example of an electrode frame 30 is illustrated in FIG. 5A. In this example, the electrode frame 30 comprises four square unit cells 200 and nine electrodes 12. In other examples, the electrode frame 30 may comprise other numbers of unit cells and electrodes, and other shapes of unit cell 200, such as a rectangle as illustrated in FIG. 4A or a parallelogram as illustrated in FIG. 4B.

FIG. 5B illustrates examples of offsets 32 which may be used to reposition the electrode frame 30. In this example, the first basis vector 201 of the unit cell is divided into N=2 sub-portions and the second basis vector 202 of the unit cell 200 is divided into N=2 sub-portions. It is possible therefore to define four different offsets for the electrode frame 30. These offsets may, for example, be defined in relation to the first basis vector 201 and the second basis vector 202 as (0, 0), (½, 0), (0, %) and (½, ½).

Thus the different offsets may be defined by a linear translation defined by a fraction of the first basis vector 201 and a fraction of the second basis vector 202.

It will be appreciated that sub-dividing the unit cell 200 by N along each basis vector produces N² different offsets. Each different offset when used to offset the electrode frame 30 defines a new different array 20 of sampling points 22.

In the example of FIG. 5B, the sub-divisions of the first basis vector 201 and the second basis vector 202 are equal (N), however, more generally, the different offsets of the electrode frame 30 may be defined by the linear translation:

n·a/N+m·b/M, where n=0,1 . . . N−1 and m=0,1 . . . M−1.

FIG. 6 illustrates an example of block 104 in FIG. 3. The figure illustrates how an array 20 of sample points 22, defined by a particular position of the electrode frame 30, is used.

For each position of the electrode frame 30 (i.e. for each different array 20 of sampling points 22) the following method may be carried out.

At block 110, an electrical input signal is provided to a pair of sampling points 22, of the array 20 of sampling points 22, for example, as illustrated in FIG. 7A or 7B. In these examples, the input signal is an electric current applied between the pair of sampling points 22.

Next at block 112, there is reception of electrical output signals from a sub-set of the other sampling points 22 of the array 20 of sampling points 22, as illustrated, for example in FIG. 7C. In the example of FIG. 7C, voltage differences between adjacent pairs of sampling points 22, are measured.

The blocks 110 and 112 are then repeated changing the input pairs of sample points 22 and the sub-set of sampling points 22.

FIG. 8 illustrates an example of how an electrode frame 30 may be repositioned without repositioning the electrode array 10. In this example, the electrode frame 30 is a sub-set of the electrode array 10. The position of the electrode frame 30 is changed by changing the sub-set of electrodes 12 of the electrode array 10. In this example, there is an electrode 12 at each possible sampling point 22. The position of the electrodes 12 in the electrode array 10 are defined by the tessellated unit cell 200 in combination with all possible offset values for the position of the tessellated unit cell.

The tessellated unit cell 200 defines the electrode frame 30 and each of the possible offset values defines a position of the electrode frame 30. Changing the offset changes which ones of the electrodes 12 are used and therefore changes the position of the electrode frame 30. It will therefore be appreciated that there is not a one-to-one mapping between the electrodes 12 of the electrode array 10 and the sampling points 22 of the array 20 of sampling points. The electrode array 10 is sub-sampled, in different ways, to produce different arrays 20 of sampling points 22.

The figure includes a legend which identifies the electrodes 12 of the electrode array 10 and uses separate indications to identify a first electrode frame 30 (first offset), a second different electrode frame 30 (second offset), a third different electrode frame 30 (third offset) and a fourth different electrode frame 30 (fourth offset).

Although the electrode frames illustrated in FIGS. 7A, 7B, 7C and 8 comprise entirely rectangular or square unit cells, other shapes are possible, such as, for example, a parallelogram as illustrated in FIG. 4B or FIG. 15A.

FIGS. 9 to 12 illustrate examples of how an electrode frame 30 comprising rectangular or square unit cells may be repositioned by repositioning the electrode array 10. In this example, there is a one-to-one mapping between electrodes 12 of the electrode array 10 and the sampling point 22 of the array 20 of sampling points 22.

The electrode frame 30 is defined by the electrodes 12 of the electrode array 10. Positioning and repositioning of the electrode frame 30 comprises physically positioning and repositioning the electrode array 10.

In this example, the tessellation of the unit cell 200 defines both the electrode frame 30 and the electrode array 10. The offsets of the unit cell represent physical shifts in the electrode array 10 and the electrode frame 30.

The control circuitry 7 in FIG. 1 may be used to control movement of the electrical array 10, for example, using a motor or a group of digital or analogue stepper motors. This may be accurate to micrometers.

FIG. 9 illustrates an example of an electrode frame 30 defined by electrodes 12. The electrode frame 30 defines an array 20 of sampling points 22 where each sampling point corresponds to an electrode 12.

FIG. 10A illustrates the use of four different offsets 32 to produce four different arrays 20 of sampling points 22. FIG. 10B illustrates all of the four arrays 20 of sampling points 22 in combination.

It will therefore be appreciated that at any point in time an array 20 of sampling points 22 as illustrated in FIG. 9 will be used. At different times different arrays 20 of sampling points 22 corresponding to the different positions of the electrode frame 30 defined by the different offsets 32 will be used and therefore, over time, the sampling points 22 illustrated in FIG. 10B will be used in the impedance imaging method.

It can be appreciated that the number and density of sampling points 22 in FIG. 10B is four times greater than the number and density of sampling points 22 in FIG. 9A. Consequently the impedance image produced using the sampling points 22 of FIG. 10B will have a higher resolution than an impedance image produced using the sampling points 22 of FIG. 9.

It should be appreciated that the order in which the different offsets 32 are implemented in FIG. 10A is such that each change in position of the electrode frame 30 involves a change only in the direction of the first basis vector 201 or the second basis vector 202 of the unit cell 200. The electrode array 10 is moved in an ordered sequence to achieve each offset 32. In this example, the first basis vector 201 and the second basis vector 202 are orthogonal.

In the example of FIG. 10A, each basis vector of the unit cell 200 is divided into two. This produces four different offsets and four different arrays 20 of sampling points 22.

In the example of FIG. 11, each basis vector is divided into three and this produces nine different offsets and consequently nine different arrays 20 of sampling points 22.

In the example of FIG. 12, each basis vector is divided into four which results in sixteen different offset values and sixteen different arrays 20 of sampling points 22.

However, it should be appreciated that each basis vector of the unit cell 200 may be divided into N (N−1 interpolations). This produces N² different offsets and N² different arrays 20 of sampling points 22.

It should be appreciated that each of the different arrays of sampling points 22 are used to obtain output electrical signals, for example as previously described in relation to FIG. 6.

FIG. 9 illustrates one example of an electrode frame 30 which is used to define an array 20 of sampling points 22. It is, however, possible to use different electrode frames 30. FIGS. 13 and 14 illustrate different electrode frames 30.

FIGS. 15, 17A and 17B illustrate an example of how an electrode frame 30 comprising parallelogram- or rhombus-shaped unit cells may be repositioned by repositioning the electrode array 10. In this example, there is a one-to-one mapping between electrodes 12 of the electrode array 10 and the sampling point 22 of the array 20 of sampling points 22.

The electrode frame 30 is defined by the electrodes 12 of the electrode array 10. Positioning and repositioning of the electrode frame 30 comprises physically positioning and repositioning the electrode array 10.

In this example, the tessellation of the unit cell 200 defines both the electrode frame 30 and the electrode array 10. The offsets of the unit cell represent physical shifts in the electrode array 10 and the electrode frame 30.

The control circuitry 7 in FIG. 1 may be used to control movement of the electrical array 10, for example, using a motor or a group of digital or analogue stepper motors. This may be accurate to micrometers.

FIG. 17A illustrates an example of an electrode frame 30 defined by electrodes 12. The electrode frame 30 defines an array 20 of sampling points 22 where each sampling point corresponds to an electrode 12.

FIG. 15 illustrates the use of four different offsets 32 to produce four different arrays 20 of sampling points 22. FIG. 17B illustrates all of the four arrays 20 of sampling points 22 in combination.

It will therefore be appreciated that at any point in time an array 20 of sampling points 22 as illustrated in FIG. 17A will be used. At different times different arrays 20 of sampling points 22 corresponding to the different positions of the electrode frame 30 defined by the different offsets 32 will be used and therefore, over time, the sampling points 22 illustrated in FIG. 17B will be used in the impedance imaging method.

It can be appreciated that the number and density of sampling points 22 in FIG. 17B is four times greater than the number and density of sampling points 22 in FIG. 17A. Consequently the impedance image produced using the sampling points 22 of FIG. 17B will have a higher resolution than an impedance image produced using the sampling points 22 of FIG. 9.

It should be appreciated that the order in which the different offsets 32 are implemented in FIG. 15 is such that each change in position of the electrode frame 30 involves a change only in the direction of the first basis vector 201 or the second basis vector 202 of the unit cell 200. The electrode array 10 is moved in an ordered sequence to achieve each offset 32. In this example, the first basis vector 201 and the second basis vector 202 are not orthogonal.

In this example, the angle between the first basis vector 201 and the second basis vector 202 is 60°.

In the example of FIG. 17A, each basis vector of the unit cell 200 is divided into two. This produces four different offsets and four different arrays 20 of sampling points 22.

However, it should be appreciated that each basis vector of the unit cell 200 may be divided into N (N−1 interpolations). This produces N2 different offsets and N2 different arrays 20 of sampling points 22.

It should be appreciated that each of the different arrays of sampling points 22 are used to obtain output electrical signals, for example as previously described in relation to FIG. 6.

FIG. 17A illustrates one example of an electrode frame 30 which is used to define an array 20 of sampling points 22. It is, however, possible to use different electrode frames 30.

Referring to FIG. 16A, implementation of the control circuitry 7 (FIG. 1) may be as a controller. The controller 7 may be implemented in hardware alone, have certain aspects in software including firmware alone or can be a combination of hardware and software (including firmware).

As illustrated in FIG. 16A the controller 7 may be implemented using instructions that enable hardware functionality, for example, by using executable computer program instructions 204 in a general-purpose or special-purpose processor 200 that may be stored on a computer readable storage medium (disk, memory etc) to be executed by such a processor 200.

The processor 200 is configured to read from and write to the memory 202. The processor 200 may also comprise an output interface via which data and/or commands are output by the processor 200 and an input interface via which data and/or commands are input to the processor 200.

The memory 202 stores a computer program 204 comprising computer program instructions (computer program code) that controls the operation of the apparatus 2 when loaded into the processor 200. The computer program instructions, of the computer program 204, provide the logic and routines that enables the apparatus to perform the methods illustrated in FIGS. 3 and 6. The processor 200 by reading the memory 202 is able to load and execute the computer program 204.

The apparatus 2 therefore comprises:

at least one processor 200; and

at least one memory 204 including computer program code 204

the at least one memory 202 and the computer program code 204 configured to, with the at least one processor 200, cause the apparatus 2 at least to perform:

using an array of sampling points defined by an electrode frame at a first position, wherein the electrode frame defines a relative displacement of sampling points; and

using a different array of sampling points defined by the same electrode frame at a different, second position.

As illustrated in FIG. 16B, the computer program 204 may arrive at the apparatus 2 via any suitable delivery mechanism 210. The delivery mechanism 210 may be, for example, a non-transitory computer-readable storage medium, a computer program product, a memory device, a record medium such as a compact disc read-only memory (CD-ROM) or digital versatile disc (DVD), an article of manufacture that tangibly embodies the computer program 204. The delivery mechanism may be a signal configured to reliably transfer the computer program 204. The apparatus 2 may propagate or transmit the computer program 204 as a computer data signal.

Although the memory 202 is illustrated as a single component/circuitry it may be implemented as one or more separate components/circuitry some or all of which may be integrated/removable and/or may provide permanent/semi-permanent/dynamic/cached storage.

Although the processor 200 is illustrated as a single component/circuitry it may be implemented as one or more separate components/circuitry some or all of which may be integrated/removable. The processor 200 may be a single core or multi-core processor.

References to ‘computer-readable storage medium’, ‘computer program product’, ‘tangibly embodied computer program’ etc. or a ‘controller’, ‘computer’, ‘processor’ etc. should be understood to encompass not only computers having different architectures such as single/multi-processor architectures and sequential (Von Neumann)/parallel architectures but also specialized circuits such as field-programmable gate arrays (FPGA), application specific circuits (ASIC), signal processing devices and other processing circuitry. References to computer program, instructions, code etc. should be understood to encompass software for a programmable processor or firmware such as, for example, the programmable content of a hardware device whether instructions for a processor, or configuration settings for a fixed-function device, gate array or programmable logic device etc.

The blocks illustrated in the FIGS. 3 and 6 may represent steps in a method and/or sections of code in the computer program 204. The illustration of a particular order to the blocks does not necessarily imply that there is a required or preferred order for the blocks and the order and arrangement of the block may be varied. Furthermore, it may be possible for some blocks to be omitted.

As used here ‘module’ refers to a unit or apparatus that excludes certain parts/components that would be added by an end manufacturer or a user. The apparatus 2 may be a module.

The term ‘comprise’ is used in this document with an inclusive not an exclusive meaning. That is any reference to X comprising Y indicates that X may comprise only one Y or may comprise more than one Y. If it is intended to use ‘comprise’ with an exclusive meaning then it will be made clear in the context by referring to “comprising only one . . . ” or by using “consisting”.

In this brief description, reference has been made to various examples. The description of features or functions in relation to an example indicates that those features or functions are present in that example. The use of the term ‘example’ or ‘for example’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’ or ‘may’ refers to a particular instance in a class of examples. A property of the instance can be a property of only that instance or a property of the class or a property of a sub-class of the class that includes some but not all of the instances in the class.

Although embodiments of the present invention have been described in the preceding paragraphs with reference to various examples, it should be appreciated that modifications to the examples given can be made without departing from the scope of the invention as claimed.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

I/we claim:

1. A method of electrical impedance imaging comprising:
using an array of sampling points defined by an electrode frame at a first position, wherein the electrode frame defines a relative displacement of sampling points; and
using a different array of sampling points defined by the same electrode frame at at least a different, second position.

2. A method as claimed in claim 1, wherein the electrode frame is defined by a tessellated unit cell of electrodes.

3. A method as claimed in claim 2, wherein the unit cell is defined by a first basis vector and a second basis vector and four electrode positions (0, 0), (1, 0), (0, 1), (1, 1) in the co-ordinate space defined by the first basis vector and the second basis vector, wherein the array of sampling points is defined by the tessellated electrode positions.

4. A method as claimed in claim 3 wherein the first basis vector and the second basis vector have the same magnitude but different directions.

5. A method as claimed in claim 1, wherein the different arrays of sampling points are defined by the same electric frame at different positional offsets.

6. A method as claimed in claim 5, wherein the different positional offsets are defined by rotation.

7. A method as claimed in claim 5, wherein the different positional offsets are defined by different translations.

8. A method as claimed in claim 7, wherein the different positional offsets are defined by different linear translations wherein each different linear translation is defined by a fraction of a first basis vector and a fraction of a second basis vector, wherein the first basis vector and the second basis vector define a unit cell of electrodes that is tessellated to form the electrode frame.

9. A method as claimed in claim 8, wherein N² different positional offsets are defined by sub-dividing the first basis vector into N first sub-portions and dividing the second basis vector into N second sub-portions and defining the linear translation by a linear combination of the one or more first sub-portions and one or more second sub-portions.

10. A method as claimed in claim 9, wherein the first sub-portions and the second sub-portions are of equal magnitude.

11. A method as claimed in claim 1, wherein the electrode frame is a sub-set of an array of electrodes, and a position of the electrode frame is changed by changing the sub-set of the array of electrodes.

12. A method as claimed in claim 11, wherein the electrode frame has a fixed arrangement of sampling points, wherein each sampling point has a fixed relative position to the other sampling points and wherein the array of sampling points is changed by changing a position of the electrode frame within the array of electrodes without changing the physical position of the array of electrodes.

13. A method as claimed in claim 1, wherein there is a one-to-one mapping between an array of electrodes and the array of sampling points.

14. A method as claimed in claim 1, wherein the electrode frame is defined by the electrodes of the electrode array and positioning of the electrode frame comprises physically positioning the electrode array.

15. A method as claimed in claim 14, wherein the electrode array is a fixed arrangement of electrodes that have a fixed relative position relative to each other.

16. A method as claimed in claim 1, wherein using an array of sampling points comprises providing an input electrical signal to a pair of sampling points; and
receiving an output electrical signal from at least some of the other sampling points.

17. A method as claimed in claim 16, wherein using an array of sampling points comprises repeatedly:
providing an input electrical signal to a pair of sampling points; and
receiving an output electrical signal from a sub-set of the other sampling points; and
changing the pair of input sampling points and/or changing the sub-set of output sampling points.

18. A method as claimed in claim 1 further comprising using electrical impedance measurements made using multiple different arrays of sampling points defined by multiple different positions of the electrode frame to produce an electrical impedance image.

19. A method as claimed in claim 18, wherein the produced electrical impedance image has a higher resolution than a resolution of the electrode frame.

20. (canceled)

21. An apparatus comprising:
at least one processor; and
at least one memory including computer program code the at least one memory and the computer program code configured to, with the at least one processor, cause the apparatus to perform:
using an array of sampling points for electrical impedance imaging defined by an electrode frame at a first position, wherein the electrode frame defines a relative displacement of sampling points; and
using a different array of sampling points for electrical impedance imaging defined by the same electrode frame at at least a different, second position.

22. (canceled)
